(19) United States
(12) Patent Application Publication
(10) Pub. No.: US 2026/0041814 A1
(43) Pub. Date: Feb. 12, 2026

(54) IMPLANTS COMPRISING A SILICONE FOAM

(71) Applicant: Elkem Silicones USA Corp., East Brunswick, NJ (US)

(72) Inventor: Kyle Edward GAINES, Ann Arbor, MI (US)

(21) Appl. No.: 18/998,552

(22) PCT Filed: Jul. 28, 2023
(86) PCT No.: PCT/US2023/071214

§ 371 Date: Jan. 27, 2025

Related U.S. Application Data
(60) Provisional application No. 63/393,084, filed on Jul. 28, 2022.

(51) Int. Cl. A61L 27/18; A61F 2/12; A61L 27/56; B29C 44/12; B29C 64/124; B29K 83/00; B33Y 80/00

(52) U.S. Cl. CPC A61L 27/18; A61F 2/12; A61L 27/56; B29C 44/1204; B29C 64/124; B33Y 80/00; B29K 2083/00

(57) ABSTRACT

The present invention relates to new implants, especially breast implants, which are bio-durable, biocompatible, which provide a soft tissue feel, and having a reduced density. Said implants comprise a shell and a filling enclosed by the shell, wherein the filling comprises a silicone foam obtained from a blowable crosslinkable silicone composition comprising an organopolysiloxane having at least two alkenyl groups bonded to silicon per molecule, an organosilicon compound having at least two and preferably at least three hydrogen atoms bonded to silicon per molecule, a hydrosilylation catalyst, at least one porogenic agent which is water, hydrogel, or an aqueous silicone emulsion, at least one chemical blowing agent, and a linear polydimethylsiloxane.

### CROSS-REFERENCE TO RELATED APPLICATIONS

The instant application claims priority to U.S. Provisional Application No. 63/393,084, filed on Jul. 28, 2022, the contents of which are hereby incorporated by reference in their entirety.

### TECHNICAL FIELD

The present invention relates to the technical field of implants. More specifically, it relates to new implants, especially breast implants, containing silicone foam, providing a soft tissue feel, and having a reduced density.

### BACKGROUND

Nowadays, reconstructive and cosmetic surgery has become a common practice as almost any part of the body can be filled to create balance and harmony. For reconstructive and cosmetic surgery, implants are used. The use of implants is forecast to a high increase due to the population aging, boost in life expectancy and style of life and improvements in implant technology. The term “implant” in this patent document means a medical device manufactured to replace a missing biological structure, support a damaged biological structure, or enhance an existing biological structure.

The implant is required to be able to provide a specific three-dimensional shape and maintain the shape for a certain period depending on the nature of the implant. The implant also needs to be bio-durable such that it is not damage by interaction with the human body; and it needs to be biocompatible. The biocompatibility of a mid-term or long-term implantable medical device refers to its ability to perform its intended function without creating any undesirable local or generalized effects.

Among all types of cosmetic and reconstructive implants, the breast implant had the largest number of implementations. Reconstructive breast surgery is practiced allowing reconstruction of a woman's breast that was affected by mastectomy, whereas cosmetic breast surgery is practiced to amend the appearance of a woman's breast, for example by adding an implant to increase the size of the breast, to correct asymmetries, change shape and fix deformities.

Besides, implants are now used more and more for other facial implants, such as brow, nose, cheek, chin and lips, and various body implants, such as tracheal stents, implantable adipose enhancements or replacements, such as gluteus maximus or cheek implants; or firmer tissues such as a calf, bicep, tricep, or abdominal muscles.

Most implants are composed of a silicone-based material which has long been recognized as one of, if not the most, biocompatible synthetic material in existence.

As a desired improvement in implant technology, it is still in need of a biocompatible light-weighting material. For example, light-weighting of breast implants is of interest to any company manufacturing said implants. Light-weighting not only improves patient wear with improved ergonomic, reducing fatigue-related issues or injury, but can decrease the amount of material needed in production of the device. This is also relevant in tissue expansion products, for instance in mastectomy procedures for those patients awaiting implants.

The patent application US 2019/0314144 A1 discloses a method for manufacturing a breast implant including producing an elastic filler material including foam, by mixing a carbonate with a hydrolyzed silicone. According to this text, carbon dioxide bubbles are produced by the reaction of the bicarbonate, such as sodium bicarbonate, with the hydrochloric acid (HCl) which is a byproduct of the hydrolysis of the silicone monomer. The blowing agent here is releasing gas bubble by acidification. Managing the acidity of the final silicone foam might be a potential risk in the context of the intended use as implant.

In the international patent application WO 2020/072374, a kit for preparing a customizable flesh simulating silicone gel or foam is disclosed. The kit according to this document can be a custom solution to improve the sensory feel of the implanted material to match the feel of their natural flesh. Said kit comprises at least 3 parts, wherein the first part contains an alkenyl groups-containing organopolysiloxane and a hydrosilylation catalyst, the second part contains an alkenyl groups-containing organopolysiloxane and a hydrogen atoms-containing organosilicon compound, and the third part contains a linear polydimethylsiloxane. According to one embodiment, the third part can further comprise a blowing agent that can generate gas by chemical decomposition or evaporation. According to another embodiment, the blowing agent can be provided to the formulation by a fourth part of the kit. Said blowing agent that off-gas at elevated temperatures can be a bicarbonate salt, in particular ammonium bicarbonate (NH₄)HCO₃, sodium bicarbonate NaHCO₃, calcium bicarbonate Ca(HCO₃)₂, or other alkali metal bicarbonates. These blowing agents release CO₂ to create the hollow cells.

Currently, there is still the need of a material for the production of implants, which is bio-durable, biocompatible, which provide a soft tissue feel, and having a reduced density.

### SUMMARY OF THE INVENTION

All these objectives, among others, are achieved by the present invention, which relates to an implant comprising a shell and a filling enclosed by the shell, wherein the filling comprises a silicone foam obtained from a blowable crosslinkable silicone composition comprising:
at least one organopolysiloxane A having at least two alkenyl groups bonded to silicon per molecule;
at least one organosilicon compound B having at least two, preferably at least three, hydrogen atoms bonded to silicon per molecule;
at least one hydrosilylation catalyst C;
at least one porogenic agent D which is water, hydrogel, or an aqueous silicone emulsion;
at least one chemical blowing agent E; and
at least one linear polydimethylsiloxane F which has a dynamic viscosity at 25° C. of between 50 mPa·s and 100000 mPa·s.

The silicone foam as defined above can be described as a “dual-blowing” silicone foam:
on one side, the porogenic agent D, which is water, hydrogel, or an aqueous silicone emulsion, generates hydrogen bubbles while incorporated into the polyaddition-crosslinking silicone composition comprising the organopolysiloxane A carrying alkenyl groups bonded to the silicon, the organosilicon compound B containing hydrogen atoms bonded to the silicon, and the hydrosilylation catalyst C, and
on the other side, the chemical blowing agent E releases gas, typically carbon dioxide, by decomposition.

The inventors discovered that the resulting dual-blowing silicone foam shows a low density, and the foam cellular structure was considerably more homogenous and stable than just using one of the blowing agents or the other, while also retaining the flesh simulating feel. It is highly suitable for being used as a filler in an implant, preferably a breast implant.

### DETAILED DESCRIPTION OF THE INVENTION

All the viscosities under consideration in the present specification correspond to a dynamic viscosity magnitude that is measured, in a manner known per se, at 25° C., at a sufficiently low shear rate gradient so that the viscosity measured with a machine of Brookfield type is independent of the rate gradient.

Unless otherwise specified, the contents in % or ppm are by weight.

The blowable crosslinkable silicone composition according to the present invention comprises at least one organopolysiloxane A having at least two alkenyl groups bonded to silicon per molecule. Preferably, the organopolysiloxane A exhibits, per molecule, at least two C₂₋₆ alkenyl groups bonded to the silicon. It can consists of at least two siloxyl units of following formula: Y^{a}R¹_{b}SiO_{(4-a-b)/2}
in which:
   Y is a C₂₋₆ alkenyl, preferably vinyl,
   R¹ is a monovalent hydrocarbon group having from 1 to 12 carbon atoms, preferably selected from the alkyl groups having from 1 to 8 carbon atoms, such as the methyl, ethyl or propyl groups, cycloalkyl groups having from 3 to 8 carbon atoms and aryl groups having from 6 to 12 carbon atoms,
   a=1 or 2, b=0, 1 or 2 and the sum a+b=2 or 3,
and optionally units of following formula: R¹_{c}SiO_{(4-c)/2}
in which R¹ has the same meaning as above and c=0, 1, 2 or 3.

Preferably, the organopolysiloxane A can have a dynamic viscosity at 25° C. of between 100 mPa·s and 120,000 mPa·s, preferably between 100 mPa·s and 80,000 mPa·s, more preferentially between 1,000 mPa·s and 50,000 mPa·s, and even more preferably between 5,000 mPa·s and 20,000 mPa·s. Said organopolysiloxane A can preferably be referred to as an organopolysiloxane oil.

The organopolysiloxane A can be a linear organopolysiloxane, a cyclic organopolysiloxane or a branched organopolysiloxane (resin). The blowable crosslinkable silicone composition according to the present invention can comprise a mixture of different organopolysiloxanes A.

According to one embodiment, the organopolysiloxane A can be a linear organopolysiloxane. Linear organopolysiloxanes exhibit a linear structure essentially formed of D or D^{Vi} siloxyl units, and of terminal M or M^{Vi} siloxyl units, with D, D^{Vi}, M and M^{Vi} defined as follows: D: R¹₂SiO_{2/2} siloxyl unit, D^{Vi}: siloxyl unit selected from the group consisting of Y₂SiO_{2/2} or YR¹SiO_{2/2} siloxyl units, M: R¹₃SiO_{1/2} siloxyl unit, M^{Vi}: siloxyl unit selected from the group consisting of the YR¹₂SiO_{1/2} and Y₂R¹SiO_{1/2}; the symbols Y and R¹ are as described above.

As examples of terminal “M or M^{Vi}” units, mention may be made of the trimethylsiloxy, dimethylphenylsiloxy, dimethylvinylsiloxy or dimethylhexenylsiloxy groups.

As examples of “D or D^{V}” units, mention may be made of the dimethylsiloxy, methylphenylsiloxy, methylvinyl-siloxy, methylbutenylsiloxy, methylhexenylsiloxy, methyldecenylsiloxy or methyldecadienylsiloxy groups.

Examples of linear or cyclic organopolysiloxanes which can be organopolysiloxane A according to the invention are:
a poly(dimethylsiloxane) comprising dimethylvinyl-silyl terminations;
a poly(dimethylsiloxane-co-methylphenylsiloxane) comprising dimethylvinylsilyl terminations;
a poly(dimethylsiloxane-co-methylvinylsiloxane) comprising dimethylvinylsilyl terminations;
a poly(dimethylsiloxane-co-methylvinylsiloxane) comprising trimethylsilyl terminations; and
a cyclic poly(methylvinylsiloxane).

Preferably, the organopolysiloxane A has a content by weight of alkenyl unit of between 0.001% and 30%, preferably between 0.01% and 10%, preferably between 0.02% and 5%.

According to a preferred embodiment, the organopolysiloxane A contains terminal dimethylvinylsilyl units, and even more preferably the organopolysiloxane A is a poly(dimethylsiloxane) comprising terminal dimethylvinylsilyl groups. The number of dimethylsiloxane units can be comprised between 5 to 1000, and preferably from 100 to 600.

According to another embodiment, the organopolysiloxane A can be a branched organopolysiloxane (i.e. a resin) comprising C₂₋₆ alkenyl units. It is preferably selected from the group consisting of the silicone resins of following formulas:
M^{Vi}Q, where the alkenyl groups bonded to silicon atoms are carried by the M groups,
MM^{Vi}Q, where the alkenyl groups bonded to silicon atoms are carried by a part of the M units,
MD^{Vi}Q, where the alkenyl groups bonded to silicon atoms are carried by the D groups,
MDD^{Vi}Q, where the alkenyl groups bonded to silicon atoms are carried by a part of the D groups,
MM^{Vi}TQ, where the alkenyl groups bonded to silicon atoms are carried by a part of the M units,
MM^{Vi}DD^{Vi}Q, where the hydrogen atoms bonded to silicon atoms are carried by a part of the M and D units,
and their mixtures,
with M, M^{Vi}, D and D^{Vi} as defined above, T: siloxyl unit of formula R¹SiO_{3/2}, and Q: siloxyl unit of formula SiO_{4/2}, where R¹ has the same meaning as above.

According to a preferred embodiment, the blowable crosslinkable silicone composition according to the present invention comprises a mixture of at least one linear organopolysiloxanes as defined above and of at least one branched organopolysiloxane (i.e. resin) as defined above. For example, the blowable crosslinkable silicone composition according to the present invention can comprise a mixture of a linear poly(dimethylsiloxane) comprising dimethylvinyl-silyl terminations and of a silicone resins of formula M^{Vi}Q, MM^{Vi}Q, MD^{Vi}Q, MDD^{Vi}Q, MM^{Vi}TQ, or MM^{Vi}DD^{Vi}Q, preferably M^{Vi}Q, MM^{Vi}Q MD^{Vi}Q, or MDD^{Vi}Q. The amount of the linear poly(dimethylsiloxane) in the blowable crosslinkable silicone composition according to the present invention can be in the range from 0.8% to 94% by weight, preferably 2.5% to 45% by weight, more preferably 3.5% to 25% by weight, of the total composition. The amount of the silicone resin in the blowable crosslinkable silicone composition according to the present invention can be in the range from 0% to 10% by weight, preferably 0.01% to 5% by weight, more preferably 0.05% to 2% by weight, of the total composition.

The blowable crosslinkable silicone composition according to the present invention further comprises at least one organosilicon compound B having at least two and preferably at least three hydrogen atoms bonded to silicon per molecule. The organosilicon compound B is preferably an organohydrogenpolysiloxane compound comprising, per molecule, at least two and preferably at least three hydrosilyl functional groups (or Si—H units).

The organosilicon compound B can advantageously be an organopolysiloxane comprising at least two, preferably at least three, siloxyl units of following formula: H_{d}R²ₑSiO_{(4-d-e)/2}
in which:
   the R² radicals, which are identical or different, represent a monovalent radical having from 1 to 12 carbon atoms,
   d=1 or 2, e=0, 1 or 2 and d+e=1, 2 or 3;
   and optionally other units of following formula: R²_{f}SiO_{(4-f)/2}
in which R² has the same meaning as above and f=0, 1, 2 or 3.

It is understood that, in the formulas above, if several R² groups are present, they can be identical to or different from one another. Preferentially, R² can represent a monovalent radical selected from the group consisting of alkyl groups having from 1 to 8 carbon atoms, optionally substituted by at least one halogen atom, such as chlorine or fluorine, cycloalkyl groups having from 3 to 8 carbon atoms and aryl groups having from 6 to 12 carbon atoms. R² can advantageously be selected from the group consisting of methyl, ethyl, propyl, 3,3,3-trifluoropropyl, xylyl, tolyl and phenyl, and most preferentially R² is methyl.

The symbol d is preferentially equal to 1.

The organosilicon compound B can exhibit a linear, branched or cyclic structure. The degree of polymerization is preferably greater than or equal to 2. Generally, it is less than 5000.

When linear polymers are concerned, the latter are essentially formed of siloxyl units selected from the units of following formulas D: R²₂SiO_{2/2} or D′: R²HSiO_{2/2} and of terminal siloxyl units selected from the units of following formulas M: R²₃SiO_{1/2} or M′: R²₂HSiO_{1/2} where R² has the same meaning as above.

Preferably, the viscosity of the organosilicon compound B is between 1 mPa·s and 5,000 mPa·s, more preferentially between 1 mPa·s and 2,000 mPa·s and more preferentially still between 5 mPa·s and 1,000 mPa·s.

Examples of organohydrogenpolysiloxanes which can be organosilicon compound B according to the invention comprising at least two hydrogen atoms bonded to a silicon atom are:
a poly(dimethylsiloxane) comprising hydrodimethyl-silyl terminations;
a poly(dimethylsiloxane-co-methylhydrosiloxane) comprising trimethylsilyl terminations;
a poly(dimethylsiloxane-co-methylhydrosiloxane) comprising hydrodimethylsilyl terminations;
a poly(methylhydrosiloxane) comprising trimethyl-silyl terminations; and
a cyclic poly(methylhydrosiloxane).

When the organosilicon compound B exhibits a branched structure, it is preferably selected from the group consisting of the silicone resins of following formulas:
M′Q, where the hydrogen atoms bonded to silicon atoms are carried by the M groups,
MM′Q, where the hydrogen atoms bonded to silicon atoms are carried by a part of the M units,
MD′Q, where the hydrogen atoms bonded to silicon atoms are carried by the D groups,
MDD′Q, where the hydrogen atoms bonded to silicon atoms are carried by a part of the D groups,
MM′TQ, where the hydrogen atoms bonded to silicon atoms are carried by a part of the M units,
MM′DD′Q, where the hydrogen atoms bonded to silicon atoms are carried by a part of the M and D units,
and their mixtures,
   with M, M′, D and D′ as defined above, T: siloxyl unit of formula R²SiO_{3/2} and Q: siloxyl unit of formula SiO_{4/2}, where R² has the same meaning as above.

Preferably, the organosilicon compound B has a content by weight of hydrosilyl Si—H functional groups of between 0.2% and 91%, more preferentially between 3% and 80% and more preferentially still between 15% and 70%.

Advantageously, the molar ratio of the hydrosilyl SiH functional groups of the organosilicon compound B to the alkene functional groups of the compound A is between 1 and 50, preferably between 2 and 30, more preferentially between 3 and 20.

According to one preferred embodiment, the blowable crosslinkable silicone composition according to the present invention comprises a mixture of at least one organosilicon compound B1 having at least three hydrogen atoms bonded to silicon per molecule and at least one organosilicon compound B2 having two hydrogen atoms bonded to silicon per molecule. Said organosilicon compound B2 contains preferably terminal dimethylhydrogensilyl units, and even more preferably the organosilicon compound B2 is a poly(dimethylsiloxane) comprising terminal dimethylhydrogensilyl groups. The number of dimethylsiloxane units within the organosilicon compound B2 can be comprised between 1 to 200, preferably between 1 and 150, and more preferably between 3 and 120. Such organosilicon compound B2 can be described as “chain extender” since it has the presumed effect of increasing the mesh size of the network when it is crosslinked. Besides, such organosilicon compound B1 having three hydrogen atoms bonded to silicon per molecule or more can be described as “crosslinker”. Preferably the organosilicon compound B1 is a poly(dimethylsiloxane-co-methylhydrosiloxane) comprising trimethylsilyl terminations and/or hydrodimethylsilyl terminations.

The hydrosilylation catalyst C can in particular be selected from platinum and rhodium compounds but also from silicon compounds, such as those described in the patent applications WO 2015/004396 and WO 2015/004397, germanium compounds, such as those described in the patent application WO 2016/075414, or nickel, cobalt or iron complexes, such as those described in the patent applications WO 2016/071651, WO 2016/071652 and WO 2016/071654. The catalyst C is preferably a compound derived from at least one metal belonging to the platinum group. These catalysts are well known. It is possible in particular to use complexes of platinum and of an organic product described in the U.S. Pat. Nos. 3,159,601, 3,159,602 and 3,220,972 and the European patents EP 0057459, EP 0188978 and EP 0190530, or the complexes of platinum and of vinylated organosiloxanes described in the U.S. Pat. Nos. 3,419,593, 3,715,334, 3,377,432 and 3,814,730.

Preferentially, the catalyst C is a compound derived from platinum. Preferentially, the catalyst C is a Karstedt platinum catalyst.

The blowable crosslinkable silicone composition according to the present invention comprises water, a hydrogel, or an aqueous silicone emulsion as porogenic agent D. The water can be added directly to the blowable crosslinkable silicone composition. Advantageously, the water can be introduced in the form of an aqueous silicone emulsion, for example a direct oil-in-water silicone emulsion or an inverse water-in-oil silicone emulsion comprising a continuous silicone oily phase, an aqueous phase and a stabilizer.

According to one embodiment, the water is introduced via an emulsion of silicone oil in water with a water content of the order of 60% by weight. When the water is introduced into the blowable crosslinkable silicone composition via an emulsion, the dispersion of the water in the blowable crosslinkable silicone composition and its stability on storage are improved.

According to one embodiment, an emulsifier can be added with the water or with the aqueous silicone emulsion. Said emulsifier can be selected by the person skilled in the art among the typical emulsifiers. It can be an anionic, cationic, amphoteric, or a nonionic emulsifier. Among these, most preferable are nonionic surfactants because they might have minimal influence on the hydrosilylation reaction. The emulsifier can be added in an amount such that the weight ratio of emulsifier vs water can be between 1:5 and 5:1, preferably between 2:1 and 1:2.

A part of the hydrosilyl functional groups of the organosilicon compound B will react with the water provided by the porogenic agent D and form the gaseous hydrogen making possible the good foaming of the composition.

The blowable crosslinkable silicone composition according to the present invention comprises at least one chemical blowing agent E. Preferably said chemical blowing agent E is at least one hydrogencarbonate salt (also commonly called “bicarbonate salt”). More preferably said chemical blowing agent E is selected from the group consisting of ammonium hydrogencarbonate (NH₄)HCO₃, sodium hydrogencarbonate NaHCO₃, calcium hydrogencarbonate Ca(HCO₃)₂, and mixtures thereof. Even more preferably said chemical blowing agent E is ammonium hydrogencarbonate.

Said chemical blowing agent E can have particles having a median particle size (D50) of ≤50 μm, and even more preferably ≤10 μm. According to a preferred embodiment, the particles of chemical blowing agent E can be grinded and sieved before use.

For the ease of application and production, the chemical blowing agent E can be pre-dispersed in said organopolysiloxane A, for example at a level from 30% to 60% by weight, with an eventual incorporation of any additive that could help to stabilize the shelf-life of the resulting composition.

The blowable crosslinkable silicone composition according to the present invention comprises at least one linear polydimethylsiloxane F which has a dynamic viscosity at 25° C. of between 50 mPa·s and 100,000 mPa·s, preferably of between 50 mPa·s to 70,000 mPa·s, more preferably of between 100 mPa·s to 20,000 mPa·s, more preferably of between 200 mPa·s to 5,000 mPa·s, even more preferably of between 1,000 mPa·s to 2,000 mPa·s.

According to a first embodiment of the invention said linear polydimethylsiloxane F has the following formula:

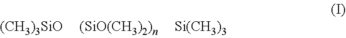

in which n is an integer from 50 to 900, and preferably from 50 to 700.

According to a second embodiment of the invention said linear polydimethylsiloxane F has the following formula:

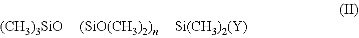

in which Y is a C₂₋₆ alkenyl, preferably vinyl, and n is an integer from 50 to 900, and preferably from 50 to 700. Said compound has exactly one alkenyl group bonded to silicon per molecule.

Preferably, the linear polydimethylsiloxane F according to the present invention consists in a mixture of the linear polydimethylsiloxanes (I) and (II) as defined above. The weight ratio of (I):(II) can be comprised between 100:0 and 0:100, or between 90:10 and 10:90, or between 80:20 and 20:80, or between 70:30 and 30:70. The blowable crosslinkable silicone composition according to the present invention can be free, or substantially free of linear polydimethylsiloxane (II). The linear polydimethylsiloxane F according to the present invention consists in one or several linear polydimethylsiloxanes (I) as defined above.

The blowable crosslinkable silicone composition according to the present invention can optionally comprise additives. Examples of suitable additives includes: a resilient additive, a reinforcement filler, a thermally or electrically conductive filler, nanoparticles, a silicone resin, a pigment, an antimicrobial agent, a UV stabilizer, a dye, a pigment, a fragrance, a flavor, an essential oil, a flame resistant additive, a thermal stabilizer, a rheology modifier, a viscosity modifier, a thickener, an adhesion promoter, a biocide, a preservative, an enzyme, a peptide, a surface-active agent, a reactive diluent, an active pharmaceutical ingredient, an excipient or a cosmetic ingredient. The content of an additive is typically below 5 wt. %, relative to the total weight of the blowable crosslinkable silicone composition, preferably below 2.5 wt. %, more preferably below 1 wt. %. The suitable additive(s) can be selected by the person skilled in the art according to the intended application and the general knowledge of the technical field.

According to one embodiment, the blowable crosslinkable silicone composition according to the present invention can optionally comprise at least one filler, preferably a reinforcing filler, which can typically improve the mechanical strength of the cured silicone elastomer article. The filler can be precipitated silica, fumed (or pyrogenic) silicas, colloidal silicas and mixtures thereof. The specific surface area of these actively reinforcing fillers ought to be at least 10 m²/g, and preferably in the range from 50 m²/g to 400 m²/g, as determined by the BET method. In a preferred embodiment, the silica reinforcing filler is fumed silica with a specific surface area of at least 10 m²/g, and preferably in the range from 50 m²/g to 400 m²/g, as determined by the BET method. Fumed silica may be used as is, in an untreated form, but is preferably subjected to hydrophobic surface treatment. The amount of the silica reinforcing filler in the blowable crosslinkable silicone composition according to the present invention can be in the range from 0% to 10% by weight, preferably 0.01% to 5% by weight, more preferably 0.05% to 2% by weight, of the total composition.

According to one embodiment, the blowable crosslinkable silicone composition according to the invention comprises (by weight, relative to the total weight of the composition):
from 1.99% to 98.99% of a partial mixture of at least one organopolysiloxane A having at least two alkenyl groups bonded to silicon per molecule, at least one organosilicon compound B having at least two and preferably at least three hydrogen atoms bonded to silicon per molecule, at least one hydrosilylation catalyst C, and at least one porogenic agent D which is water, hydrogel, or an aqueous silicone emulsion;
from 0.01% to 2% of at least one chemical blowing agent E; and
from 1% to 98% of at least one linear polydimethylsiloxane F which has a dynamic viscosity at 25° C. of between 50 mPa·s and 100000 mPa·s.

According to another embodiment, the blowable crosslinkable silicone composition according to the invention comprises (by weight, relative to the total weight of the composition):
from 4.95% to 49.95% of a partial mixture of at least one organopolysiloxane A having at least two alkenyl groups bonded to silicon per molecule, at least one organosilicon compound B having at least two and preferably at least three hydrogen atoms bonded to silicon per molecule, at least one hydrosilylation catalyst C, and at least one porogenic agent D which is water, hydrogel, or an aqueous silicone emulsion;
from 0.05% to 1.5% of at least one chemical blowing agent E; and
from 50% to 95% of at least one linear polydimethylsiloxane F which has a dynamic viscosity at 25° C. of between 50 mPa·s and 100000 mPa·s.

According to another embodiment, the blowable crosslinkable silicone composition according to the invention comprises (by weight, relative to the total weight of the composition):
from 6.9% to 24.9% of a partial mixture of at least one organopolysiloxane A having at least two alkenyl groups bonded to silicon per molecule, at least one organosilicon compound B having at least two and preferably at least three hydrogen atoms bonded to silicon per molecule, at least one hydrosilylation catalyst C, and at least one porogenic agent D which is water, hydrogel, or an aqueous silicone emulsion;
from 0.1% to 1.0% of at least one chemical blowing agent E; and
from 75% to 93% of at least one linear polydimethylsiloxane F which has a dynamic viscosity at 25° C. of between 50 mPa·s and 100000 mPa·s.

Within the above mentioned embodiments, the partial mixture of at least one organopolysiloxane A having at least two alkenyl groups bonded to silicon per molecule, at least one organosilicon compound B having at least two and preferably at least three hydrogen atoms bonded to silicon per molecule, at least one hydrosilylation catalyst C, and at least one porogenic agent D which is water, hydrogel, or an aqueous silicone emulsion, can have the following composition (by weight, relative to the total weight of the partial mixture):
from 40% to 95% of at least one organopolysiloxane A having at least two alkenyl groups bonded to silicon per molecule;
from 1% to 20% of at least one organosilicon compound B having at least two and preferably at least three hydrogen atoms bonded to silicon per molecule;
from 2 to 400 ppm, of at least one platinum hydrosilylation catalyst C, calculated as weight of platinum metal;
from 0.3% to 2.5% of at least one porogenic agent D which is water, hydrogel, or an aqueous silicone emulsion More preferentially, said partial mixture can have the following composition (by weight, relative to the total weight of the partial mixture):
from 50% to 90% of at least one organopolysiloxane A having at least two alkenyl groups bonded to silicon per molecule;
from 3% to 15% of at least one organosilicon compound B having at least two and preferably at least three hydrogen atoms bonded to silicon per molecule;
from 5 ppm and 200 ppm, of at least one platinum hydrosilylation catalyst C, calculated as weight of platinum metal;
from 0.5% to 1.5% of at least one porogenic agent D which is water, hydrogel, or an aqueous silicone emulsion.

Another object of the invention concerns a process for preparing the implant as disclosed above. Said implant comprises a shell and a filling enclosed by the shell. According to one embodiment, the filling enclosed by the shell consists in, or essentially consists in, the silicone foam according to the present invention. According to other embodiments, in addition to the foam, the filling may comprise other elements, including flexible bodies, for example, air-containing, or gas-containing, bodies. According to one embodiment, the filling enclosed by the shell comprises hollow microspheres. As examples of suitable hollow microspheres, it can be cited hollow glass microspheres or hollow ceramic microspheres.

Hollow glass microspheres are sometimes termed “hollow glass beads” or “hollow glass bubbles”. They are small hollow spheres of hardened silica (glass) that can vary in size and density depending on the grade. They have a shell that is thick enough to maintain structural rigidity. Due to their hollow nature, they are very lightweight, with a density that varies with size and wall thickness. In bulk they appear as a white powder. The main differences between grades are in their size, strength and density, with the strength of the microspheres being expressed in terms of their average isostatic crushing strength.

According to an embodiment, hollow glass beads are hollow borosilicate glass microspheres.

According to an embodiment, the hollow glass microspheres have a true density ranging from 0.10 g/cm³ (gram per cubic centimeter) to 0.75 g/cm³.

The terms “true density” is the quotient obtained by dividing the mass of a sample of hollow glass microspheres by the true volume of that mass of glass bubbles as measured by a gas pycnometer. The “true volume” is the aggregate total volume of the glass bubbles, not the bulk volume.

According to a preferred embodiment, hollow glass microspheres are selected from:
1. 3M™ Glass Bubbles Floated Series (A16/500, G18, A20/1000, H20/1000, D32/4500 and H50/10,000EPX glass bubbles products) and 3M™ Glass Bubbles K, S, iM and XLD Series (such as but not limited to K1, K11, K15, S15, S22, K20, K20HS, K25, S32, S32LD, S35, XLD3000, S28HS, S35, K37, S38, S38HS, S38XHS, S32HS, K46, K42HS, S42XHS, S60, S60HS, iM16K, iM30K glass bubbles products) sold by 3M Company. Said glass bubbles exhibit various crush strengths ranging from 1.72 MPa (250 psi) to 186.15 MPa (27,000 psi) at which ten percent by volume of the first plurality of glass bubbles collapses. Other glass bubbles sold by 3M such as 3M™ Glass Bubbles—HGS Series and 3M™ Glass Bubbles with Surface Treatment could also be used.
2. Hollow glass microspheres sold under the tradename SPHERICEL® (products such as: 110P8, 60P18, 34P30, and 25P45) or under the tradename Q-Cel® Lightweight (products such as: 6014, 6019, 7019, 6019S, 5020, 5020FPS, 7023, 7028, 2058, 6036, 7037, 7040S, 6042S, 6048 and 5070S), sold by Potters Industries Inc.

Suitable hollow glass microspheres are not surface-treated or are surface-treated. Surface-treated hollow glass microsphere can be typically hydrophobic. Surface-treatment agents can be for instance silane coupling agent such as: aminopropyltriethoxysilane, γ-glycidoxypropyltrimethoxysilane, γ-(methacryloloxy)propyltrimethoxysilane (also known as silane coupling agent KH-570) and sodium methylsiliconate.

Hollow ceramic microspheres also referred to as cenospheres are lightweight, inert, hollow sphere filled with inert air or gas, typically produced as a byproduct of coal combustion at thermal power plants. They are made largely of silica and alumina. The color of cenospheres varies from gray to almost white and their density is about 0.4 g/cm³ to 0.8 g/cm³. It flows like a liquid, with the appearance of a powder. Suitable cenospheres are not surface-treated or are surface-treated with a silane-based coupling agent such as one or more of 3-aminopropyltriethoxysilane, gamma-glycidoxypropyltrimethoxysilane, gamma-(methacryloyloxy) propyltrimethoxysilane, 3-aminopropyltrimethoxysilane, 4-aminopropylmethyldimethoxysilane or 3-aminopropylmethyldiethoxysilane.

Commercially available examples of hollow ceramic microspheres are Z-Light™ Spheres Microspheres commercialized by 3M™ (products such as: 3M™ Z-Light™ Spheres G-3125, G-3150 and G-3500).

According to a first embodiment, an object of the invention is a process for preparing an implant comprising a shell and a filling enclosed by the shell, comprising the steps of:
1a) combining the components of the blowable crosslinkable silicone composition, according to the invention and as defined above, to provide a filling precursor of a silicone foam,
1b) fill said filling precursor of a silicone foam into the shell, and
1c) allowing said filling precursor of a silicone foam to blow and crosslink, to provide the implant.

A suitable shell may be selected by the person skilled in the art according to the common knowledge of the technical field. The shell can be for instance a silicone elastomer, preferably a vulcanized silicone rubber, which can be single- or multi-layered, smooth or textured, barrier-coated, or covered with polyurethane foam. Conventional breast implant shells are multi-layered or laminated. Specifically, such shells include outer “rupture-resistant” layers, and an inner “barrier” layer, sandwiched between the outer layers and effective to resist silicone bleed. For example, it can include a low diffusion silicone elastomer shell made with outer layers of a dimethyl-diphenyl silicone elastomer, having a diphenyl polymer mole percent of 5%, and a barrier layer of dimethyl-diphenyl silicone elastomer having a diphenyl polymer mole percent of 15%. Another suitable example of flexible shell that can be used according to the invention is a flexible shell including a substantially homogenous layer enveloping and in direct contact with the silicone gel of the core, made of a silicone elastomer comprising a polydimethylsiloxane backbone having diphenyl pendant groups such as the mole percent of said diphenyl siloxane units is about 15%.

Flexible implant shells can be manufactured by a conventional dip-molding process or spray process. Dip-molding process involves typically dipping a suitably shaped mandrel into a silicone elastomer dispersion, whereas spraying process consists in spraying said silicone elastomer dispersion onto suitably shaped mandrel. Afterwards, the shell is peeled from the mandrel and a shell hole resulting from the molding process is patched. The hollow interior of the shell can then be filled with the filling precursor according to the invention, by means of an aperture in the patch. The aperture in the patch is then sealed with a silicone adhesive and the prosthesis is heat cured. Another process for forming implant shells is rotational molding, such as the system and methods described in U.S. Pat. No. 6,602,452. Alternatively, shells can be manufactured by injection-molding.

According to another embodiment, an object of the invention is a process for preparing an implant comprising a shell and a filling enclosed by the shell, comprising the steps of:
2a) combining the components of the blowable crosslinkable silicone composition, according to the invention and as defined above, to provide a filling precursor of a silicone foam,
2b) allowing said filling precursor of a silicone foam to blow and crosslink, and
2c) creating the shell around said silicone foam, to provide the implant.

According to said embodiment, step 2b) can preferably be performed into a mold so that the silicone foam may have the specific desired geometry.

The shell can be obtained in step 2c) according to the methods as known by the person skilled in the art of the technical field, as disclosed above, but using the silicone foam obtained at step 2b) instead of the suitably shaped mandrel. For example, step 2c) can preferably be performed by dipping the silicone foam obtained at step 2b) into a silicone elastomer dispersion. Alternatively, step 2c) can preferably be performed by spraying said silicone elastomer dispersion onto the silicone foam obtained at step 2b).

Step 1c) and step 2b) of the process according to the invention (i.e. blowing and crosslinking the filling precursor into a silicone foam) can be carried out by heating at a temperature range of between 50° C. to 200° C., preferably of between 100° C. to 170° C. The temperature of step c) can be adapted according to the degradation temperature of the chemical blowing agent E. For example, for ammonium hydrogencarbonate, degradation temperature is around 60° C. The temperature of step 1c) and step 2b) can be preferably set above 60° C., so that both blowing and crosslinking can proceed essentially simultaneously. Alternatively, step 1c) and step 2b) can start at room temperature, and the temperature can be raised afterwards so as to control separately blowing by the chemically blowing agent E and crosslinking.

Another objective of the invention is to provide a new process for additive manufacturing a 3D-shape article made of said silicone foam. Such process will also enable to manufacture complex shape objects made of such biocompatible materials.

One object of the present invention relates to a process for additive manufacturing a 3D-shape article made of a silicone foam, in particular for use in medical devices, comprising the steps of:
3a) printing with a 3D printer a portion of said blowable crosslinkable silicone composition as defined above, to form a deposit into a supporting material SM which is a gel or microgel suitable for 3D-gel printing silicone foam, said deposit is achieved by way of a device which has at least one delivery unit which can be positioned in x-, y- and z-directions,
3b) allowing the printed blowable crosslinkable silicone composition to partially or totally blow and crosslink, to obtain a silicone foam deposit within said supporting material SM,
3c) optionally repeating several times steps a) and b) until the desired 3D-shape is obtained,
3d) removing mechanically or via dissolution in a solvent said supporting material SM, and
3e) recovering a 3D-shape article made of a silicone foam.

According to a specific embodiment, step 3b) can be carried out by heating at a temperature range of between 50° C. to 200° C., preferably of between 100° C. to 170° C. The temperature of step 3b) can be adapted according to the degradation temperature of the chemical blowing agent E. For example, for ammonium hydrogencarbonate, degradation temperature is around 60° C. The temperature of step 3b) can be preferably set above 60° C., so that both blowing and crosslinking can proceed essentially simultaneously. Alternatively, step 3b) can start at room temperature, and the temperature can be raised afterwards so as to control separately blowing by the chemically blowing agent E and crosslinking. This specific embodiment could advantageously provide de silicone foam having anisotropic properties.

Printing is preferably carried out layer by layer with a 3D-printer which may be selected from an extrusion 3D printer or a material jetting 3D printer. 3D printing is generally associated with a host of related technologies used to fabricate physical objects from computer generated, e.g. computer-aided design (CAD), data sources. “3D printer” is defined as a machine used for 3D printing, and “3D printing” is defined as the fabrication of objects through the deposition of a material using a print head, nozzle, or another printer technology.

In one preferred embodiment, the method for manufacturing article made of silicone foam according to the invention uses an extrusion 3D printer. The blowable crosslinkable silicone composition is extruded through a nozzle. The nozzle may be heated to aid in dispensing the addition crosslinking silicone composition. The blowable crosslinkable silicone composition to be dispensed through the nozzle may be supplied from a cartridge-like system. It is also possible to use a coaxial cartridges system with a static mixer and only one nozzle. Pressure will be adapted to the fluid to be dispensed, the associated nozzle average diameter and the printing speed. Because of the high shear rate occurring during the nozzle extrusion, the viscosity of the blowable crosslinkable silicone composition is greatly lowered and so permits the printing of fine layers. Cartridge pressure could vary from 1 bar (i.e. atmospheric pressure) to 28 bars, preferably from 1 bar to 10 bars and most preferably from 2 bars to 8 bars. An adapted equipment using aluminum cartridges can be used to resist such a pressure. The nozzle and/or build platform moves in the x-y (horizontal plane) to complete the cross section of the object, before moving in the z-axis (vertical) plane once one layer is complete. The nozzle has a high x-y-z-movement precision around 10 pm. After each layer is printed in the x- and y-work plane, the nozzle is displaced in the z-direction only far enough that the next layer can be applied in the x-, y-work place. In this manner, the 3D article is built one layer at a time from the bottom to the upward. The average diameter of a nozzle is related to the thickness of the layer. In an embodiment, the diameter of the layer is comprised from 50 pm to 2000 pm, preferably from 100 pm to 800 pm and most preferably from 100 pm to 500 pm. Advantageously, printing speed is comprised between 1 mm/s and 50 mm/s, preferably between 5 mm/s and 30 mm/s to obtain the best compromise between good accuracy and manufacture speed.

The supporting material SM is a gel or microgel suitable for 3D-gel printing silicone foam. The gel or microgel provides a constant support for the liquid material during 3D-printing. This allows more complex objects to be printed without the need for added supports, and at a faster pace. The supporting material SM may be selected by the person skilled in the art among the materials publicly disclosed, for instance in the international patent applications WO 2019/215190 A1 and WO 2020/127882 A1, or the US patent applications US 2015/0028523 A1, US 2018/0036953 A1, and US 2018/0057682 A1. Further details could also be found in the scientific publication of Arthur Colly, Christophe Marquette, and Edwin-Joffrey Courtial: “Poloxamer/Poly(ethylene glycol) Self-Healing Hydrogel for High-Precision Freeform Reversible Embedding of Suspended Hydrogel” (Langmuir 2021, 37, 14, 4154-4162).

According to one embodiment of the claimed process, the supporting material SM may be provided as a matrix, for example into a container, and placed at a required temperature. According to another embodiment, the supporting material SM may be delivered simultaneously or at staggered intervals with the blowable crosslinkable silicone composition, at a specific location by way of a device which has at least one delivery unit which can be positioned in x-, y- and z-directions.

The 3D-shape article made of a silicone foam as recovered from step 3e) can advantageously be used as medical device. In the framework of the present invention, said 3D-shape article made of a silicone foam can be used as an implant, preferably as a breast implant.

According to a specific embodiment, an object of the invention is a process for preparing an implant comprising a shell and a filling enclosed by the shell, comprising the steps of providing a 3D-shape article made of a silicone foam according to the process as disclosed above, and creating the shell around said silicone foam, to provide the implant.

Alternatively, the silicone foam according to the invention and the shell can be both simultaneously 3D-printed by using specifically designed 3D-printer equipped with a double-nozzle system, able to 3D-print at the same time shell and filler.

After finishing the implant according to the present invention, the steps required to make a finished product may be similar to those known in the art. For example, the filled implant is packaged, and the packaged implant sterilized.

The implant according to the present invention can be a breast implant, a facial implant, such as brow, nose, cheek, chin and lips, or another body implant, such as tracheal stents, implantable adipose enhancements or replacements, such as gluteus maximus, breast or cheek implants; or firmer tissues such as a calf, bicep, triceps, or abdominal muscles. Preferably the implant according to the present invention is a breast implant.

Various embodiments of the present invention can be better understood by reference to the following examples which are offered by way of illustration. The present invention is not limited to the examples given herein.

### EXAMPLES

### Raw Materials:

Organopolysiloxane A1=mixture of several polydimethylsiloxane oils with dimethylvinylsilyl end-units with viscosity at 25° C. varying from about 4,000 mPa to about 100,000 mPa·s, and having an average viscosity at 25° C. of about 10,000 mPa·s.

Organopolysiloxane A2=MD^{Vi}Q branched organopolysiloxane, where the vinyl groups bonded to silicon atoms are carried by the D groups

Organopolysiloxane B1=poly(methylhydrogen)siloxane with trimethylsilyl end-units with a viscosity at 25° C. of about 20 mPa·s

Organopolysiloxane B2=polydimethylsiloxane with dimethylhydrogensilyl end-units with a viscosity at 25° C. of about 7 mPa·s

Catalyst C=10% by weight of Platinum metal, known as Karstedt's catalyst

Emulsion D=silicone emulsion containing about 59.5 wt. % of water

Blowing agent E1=Ammonium hydrogencarbonate

Blowing agent E2=Finely grounded ammonium hydrogencarbonate mixed with silica and polydimethylsiloxane oil (NH₄HCO₃ content=50 wt. %)

Polydimethylsiloxane F1=PDMS with a viscosity at 25° C. of about 1000 mPa·s

Polydimethylsiloxane F2=PDMS with a viscosity at 25° C. of about 5000 mPa·s

Polydimethylsiloxane F3=mixture of several polydimethylsiloxane oils (PDMS and polydimethylsiloxanes with one dimethylvinylsilyl end-unit and one trimethylsilyl end-unit) having an average viscosity at 25° C. of about 300 mPa·s

Polydimethylsiloxane F4=mixture of several polydimethylsiloxane oils (PDMS and polydimethylsiloxanes with one dimethylvinylsilyl end-unit and one trimethylsilyl end-unit) having an average viscosity at 25° C. of about 1000 mPa·s

Polydimethylsiloxane F5=mixture of several polydimethylsiloxane oils (PDMS and polydimethylsiloxanes with one dimethylvinylsilyl end-unit and one trimethylsilyl end-unit) having an average viscosity at 25° C. of about 2000 mPa·s

Polydimethylsiloxane F6=mixture of several polydimethylsiloxane oils (PDMS and polydimethylsiloxanes with one dimethylvinylsilyl end-unit and one trimethylsilyl end-unit) having an average viscosity at 25° C. of about 20,000 mPa·s

### Comparative Example 1

A blowable crosslinkable silicone composition was prepared by mixing the components as mentioned in Table 1 below:

TABLE 1

| Comparative Example 1 | |
|---|---|
| Organopolysiloxane A1 | 65.50% |
| Organopolysiloxane A2 | 20.25% |
| Organopolysiloxane B1 | 9.50% |
| Organopolysiloxane B2 | 3.70% |
| Emulsion D | 1.00% |
| Catalyst C | 0.05% |

The obtained hydrogen-blown silicone foam shows a high stiffness. The feel is not enough similar to human tissues.

### Comparative Example 2

The silicone foam disclosed in prior art document WO 2020/072374 in Table 14 was reproduced. The raw materials (linear PDMS, polydimethylsiloxane with dimethylvinylsilyl end-units, platinum catalyst, crosslinker and ammonium hydrogencarbonate) were mixed and temperature was maintained at 150° C. for 30 minutes so that the crosslinking and the blowing occurred. The silicone foam is exclusively chemically foamed.

The feel of this material was greatly improved in comparison to the Comparative example 1, but it was found that the material had a very irregular cell structure which caused manufacturing concerns as the uniform flesh feel of the device.

### Examples 1-6

Blowable crosslinkable silicone compositions were prepared by mixing the components as mentioned in Table 2 below. After mixing, the temperature was maintained at 150° C. for 60 minutes.

TABLE 2

| | Ex. 1 | Ex. 2 | Ex.3 | Ex. 4 | Ex. 5 | Ex. 6 |
|---|---|---|---|---|---|---|
| Hydrogen-blown | 10% | 15% | 7% | 10% | 15% | 7% |
| silicone foam | | | | | | |
| composition | | | | | | |
| according to | | | | | | |
| Comparative | | | | | | |
| Example 1 | | | | | | |
| Blowing | 0.125% | 0.125% | 0.125% | 0.25% | 0.25% | 0.25% |
| agent E1 | | | | | | |
| Polydimethyl- | 90% | 85% | 93% | | | |
| siloxane F1 | | | | | | |
| Polydimethyl- | | | | 90% | 85% | 93% |
| siloxane F2 | | | | | | |

The silicone foams obtained according to the invention, which are simultaneously hydrogen-blown and chemically blown foams, show low density, low weight, good cell structure and with natural tissue-like feel.

### Examples 7-9

Blowable crosslinkable silicone compositions were prepared by mixing the components as mentioned in Table 3 below. After mixing, the temperature was maintained at 150° C. for 60 minutes.

TABLE 3

| | Ex. 1 | | | |
|---|---|---|---|---|
| | (from | | | |
| | Table 2) | Ex. 7 | Ex. 8 | Ex. 9 |
| Hydrogen- | 10% | 10% | 10% | 10% |
| blown silicone | | | | |
| foam composition | | | | |
| according to | | | | |
| Comparative | | | | |
| Example 1 | | | | |
| Blowing agent E1 | 0.125% | | | |
| Blowing agent E2 | | 0.25% | 0.25% | 0.25% |
| Polydimethylsiloxane F1 | 90% | 89.75% | 44.875% | |
| Polydimethylsiloxane F3 | | | 44.875% | 89.75% |

### Examples 10-15

General composition of the hydrogen-blown silicone foam “H2B foam”:
67.10 wt. % of a mixture of several polydimethylsiloxane oils with dimethylvinylsilyl end-units, having an average viscosity at 25° C. of about 1,000 mPa·s;
11.2 wt. % of treated fumed silica;
0.64 wt. % of water;
0.64 wt. % of an emulsifier;
0.03 wt. % of Karstedt's catalyst (containing 10% by weight of Platinum metal);
20.00 wt. % of poly(methylhydrogen)siloxane with trimethylsilyl end-units with a viscosity at 25° C. of about 20 mPa·s;
0.39 wt. % of ethynylcyclohexanol.

Blowable crosslinkable silicone compositions were prepared by mixing the components as mentioned in Table 4 below. After mixing, the temperature was maintained at 150° C. for 60 minutes.

TABLE 4

| | Ex. 10 | Ex. 11 | Ex. 12 | Ex. 13 | Ex. 14 | Ex. 15 |
|---|---|---|---|---|---|---|
| H2B foam with | 6.27% | | | 6.27% | 6.27% | 6.27% |
| emulsifier = | | | | | | |
| PVP10 | | | | | | |
| H2B foam with | | 6.27% | | | | |
| emulsifier = | | | | | | |
| PVP40 | | | | | | |
| H2B foam with | | | 6.27% | | | |
| emulsifier = | | | | | | |
| silicone | | | | | | |
| polyether | | | | | | |
| Blowing | 0.25% | 0.25% | 0.25% | 0.25% | 0.25% | 0.25% |
| agent E2 | | | | | | |
| Polydimethyl- | 93.48% | 93.48% | 93.48% | | | |
| siloxane F3 | | | | | | |
| Polydimethyl- | | | | 93.48% | | |
| siloxane F4 | | | | | | |
| Polydimethyl- | | | | | 93.48% | |
| siloxane F5 | | | | | | |
| Polydimethyl- | | | | | | 93.48% |
| siloxane F6 | | | | | | |

PVP10=polyvinylpyrrolidone; average molecular weight 10,000
PVP40=polyvinylpyrrolidone; average molecular weight 40,000

### Example 16

Example 10 was reproduced, except that the composition of the hydrogen-blown silicone foam “H2B foam” comprised 14.6 wt. % of treated fumed silica (instead of 11.2 wt. %) and 63.7 wt. % of the same mixture of several polydimethylsiloxane oils (instead of 67.1 wt. %).

The silicone foams obtained with the compositions of Examples 7 to 16 show low density, low weight, and good cell structure.

| | Relative density |
|---|---|
| | (at room temperature) |
| Ex. 9 | 0.7450 |
| Ex. 10 | 0.7489 |
| Ex. 11 | 0.7878 |
| Ex. 13 | 0.7282 |
| Ex. 14 | 0.6070 |
| Ex. 15 | 0.6589 |
| Ex. 16 | 0.9062 |

1. An implant comprising a shell and a filling enclosed by the shell, wherein the filling comprises a silicone foam obtained from a blowable crosslinkable silicone composition comprising:
at least one organopolysiloxane A having at least two alkenyl groups bonded to silicon per molecule;
at least one organosilicon compound B having at least two and optionally at least three hydrogen atoms bonded to silicon per molecule;
at least one hydrosilylation catalyst C;
at least one porogenic agent D which is water, hydrogel, or an aqueous silicone emulsion;
at least one chemical blowing agent E; and
at least one linear polydimethylsiloxane F which has a dynamic viscosity at 25° C. of between 50 mPa·s and 100000 mPa·s.

2. The implant according to claim 1, wherein the organopolysiloxane A is a linear organopolysiloxane; optionally the organopolysiloxane A contains terminal dimethylvinylsilyl units, and optionally the organopolysiloxane A is a poly(dimethylsiloxane) comprising terminal dimethylvinylsilyl groups.

3. The implant according to claim 1, wherein the organopolysiloxane A is a branched organopolysiloxane comprising C₂₋₆ alkenyl units; optionally selected from the group consisting of the silicone resins of following formulas:
M^{Vi}Q, where the alkenyl groups bonded to silicon atoms are carried by the M groups,
MM^{Vi}Q, where the alkenyl groups bonded to silicon atoms are carried by a part of the M units,
MD^{Vi}Q, where the alkenyl groups bonded to silicon atoms are carried by the D groups,
MDD^{Vi}Q, where the alkenyl groups bonded to silicon atoms are carried by a part of the D groups,
MM^{Vi}TQ, where the alkenyl groups bonded to silicon atoms are carried by a part of the M units,
MM^{Vi} DD^{Vi}Q, where the hydrogen atoms bonded to silicon atoms are carried by a part of the M and D units,
and/or a mixture thereof,
with M: R¹₃SiO_{1/2} siloxyl unit, M^{Vi}: siloxyl unit selected from the group consisting of the YR¹₂SiO_{1/2} and Y₂R¹SiO_{1/}, D: R¹₂SiO_{2/2} siloxyl unit, D^{Vi}: siloxyl unit selected from the group consisting of Y₂SiO_{2/2} or YR¹SiO_{2/2} siloxyl units, T: siloxyl unit of formula R¹SiO_{3/2}, and Q: siloxyl unit of formula SiO_{4/2}; the symbol Y is a C₂₋₆ alkenyl, optionally vinyl; and the symbol R¹ is a monovalent hydrocarbon group having from 1 to 12 carbon atoms, optionally selected from the alkyl groups having from 1 to 8 carbon atoms, optionally the methyl, ethyl or propyl groups, cycloalkyl groups having from 3 to 8 carbon atoms and aryl groups having from 6 to 12 carbon atoms.

4. The implant according to claim 1, wherein the blowable crosslinkable silicone composition comprises a mixture of at least one linear organopolysiloxane having at least two alkenyl groups bonded to silicon per molecule and of at least one branched organopolysiloxane having at least two alkenyl groups bonded to silicon per molecule.

5. The implant according to claim 1, wherein the blowable crosslinkable silicone composition comprises a mixture of at least one organosilicon compound B1 having at least three hydrogen atoms bonded to silicon per molecule and at least one organosilicon compound B2 having two hydrogen atoms bonded to silicon per molecule.

6. The implant according to claim 1, wherein the porogenic agent D is an aqueous silicone emulsion.

7. The implant according to claim 1, wherein the porogenic agent D is a mixture of water and at least one emulsifier.

8. The implant according to claim 1, wherein the chemical blowing agent E is at least one hydrogencarbonate salt, optionally selected from the group consisting of ammonium hydrogencarbonate (NH₄)HCO₃, sodium hydrogencarbonate NaHCO₃, calcium hydrogencarbonate Ca(HCO₃)₂, and mixtures thereof, and optionally the chemical blowing agent E is ammonium hydrogencarbonate.

9. The implant according to claim 1, wherein the linear polydimethylsiloxane F is in a mixture of linear polydimethylsiloxanes (I) and (II), with a weight ratio of (I):(II) comprised between 100:0 and 0:100,
wherein the linear polydimethylsiloxane (I) has the following formula:
(CH₃)₃SiO(SiO(CH₃)₂)ₙSi(CH₃)₃ (I)
in which n is an integer from 50 to 900, and optionally from 50 to 700; and
wherein the linear polydimethylsiloxane (II) has the following formula:
(CH₃)₃SiO(SiO(CH₃)₂)ₙSi(CH₃)₂(Y) (II)
in which Y is a C₂₋₆ alkenyl, optionally vinyl, and n is an integer from 50 to 900, and optionally from 50 to 700.

10. The implant according to claim 1, wherein the blowable crosslinkable silicone composition comprises (by weight, relative to the total weight of the composition):
from 1.99% to 98.99% of a partial mixture of at least one organopolysiloxane A having at least two alkenyl groups bonded to silicon per molecule, at least one organosilicon compound B having at least two and optionally at least three hydrogen atoms bonded to silicon per molecule, at least one hydrosilylation catalyst C, and at least one porogenic agent D which is water, hydrogel, or an aqueous silicone emulsion;
from 0.01% to 2% of at least one chemical blowing agent E; and
from 1% to 98% of at least one linear polydimethylsiloxane F which has a dynamic viscosity at 25° C. of between 50 mPa·s and 100000 mPa·s.

11. The implant according to claim 10, wherein a partial mixture of at least one organopolysiloxane A having at least two alkenyl groups bonded to silicon per molecule, at least one organosilicon compound B having at least two and optionally at least three hydrogen atoms bonded to silicon per molecule, at least one hydrosilylation catalyst C, and at least one porogenic agent D which is water, hydrogel, or an aqueous silicone emulsion, has the following composition (by weight, relative to the total weight of the partial mixture):
from 40% to 95% of at least one organopolysiloxane A having at least two alkenyl groups bonded to silicon per molecule;
from 1% to 20% of at least one organosilicon compound B having at least two and optionally at least three hydrogen atoms bonded to silicon per molecule;
from 2 and 400 ppm, of at least one platinum hydrosilylation catalyst C, calculated as weight of platinum metal;
from 0.3% to 2.5% of at least one porogenic agent D which is water, hydrogel, or an aqueous silicone emulsion.

12. The implant according to claim 1, wherein the filling enclosed by the shell comprises the silicone foam and further comprises hollow microspheres, optionally hollow glass microspheres.

13. The implant according to claim 1, wherein said implant is a breast implant, a facial implant, optionally brow, nose, cheek, chin and lips, or another body implant, optionally tracheal stents, implantable adipose enhancements or replacements, optionally gluteus maximus or cheek implants; or firmer tissues optionally a calf, bicep, triceps, or abdominal muscles; and optionally the implant is a breast implant.

14. A process for preparing an implant of claim 1 comprising a shell and a filling enclosed by the shell, comprising:
1a) combining components of the blowable crosslinkable silicone composition of the implant, to provide a filling precursor of a silicone foam,
1b) filling said filling precursor of a silicone foam into the shell, and
1c) allowing said filling precursor of a silicone foam to blow and crosslink, to provide the implant.

15. A process for preparing an implant of claim 1 comprising a shell and a filling enclosed by the shell, comprising:
2a) combining components of the blowable crosslinkable silicone composition, to provide a filling precursor of a silicone foam,
2b) allowing said filling precursor of a silicone foam to blow and crosslink, and
2c) creating the shell around said silicone foam, to provide the implant.

16. A process for additive manufacturing a 3D-shape article made of a silicone foam, optionally for a medical device optionally for an implant, comprising:
3a) printing with a 3D printer a portion of blowable crosslinkable silicone composition comprising
at least one organopolysiloxane A having at least two alkenyl groups bonded to silicon per molecule;
at least one organosilicon compound B having at least two and optionally at least three hydrogen atoms bonded to silicon per molecule;
at least one hydrosilylation catalyst C;
at least one porogenic agent D which is water, hydrogel, or an aqueous silicone emulsion;
at least one chemical blowing agent E; and
at least one linear polydimethylsiloxane F which has a dynamic viscosity at 25° C. of between 50 mPa·s and 100000 mPa·s to form a deposit into a supporting material SM which is a gel or microgel suitable for 3D-gel printing silicone foam, said deposit is achieved by way of a device which has at least one delivery unit which can be positioned in x-, y- and z-directions,
3b) allowing the printed blowable crosslinkable silicone composition to partially or totally blow and crosslink, to obtain a silicone foam deposit within said supporting material SM,
3c) optionally repeating several times a) and b) until a desired 3D-shape is obtained,
3d) removing mechanically or via dissolution in a solvent said supporting material SM, and
3e) recovering a 3D-shape article made of a silicone foam.
